(19) United States
(12) Patent Application Publication
(10) Pub. No.: US 2022/0240844 A1
(43) Pub. Date: Aug. 4, 2022

(54) Evaluating Rehabilitation Potential in Pain Patients

(71) Applicant: David Bruce Ross, Southwest Ranches, FL (US)

(72) Inventor: David Bruce Ross, Southwest Ranches, FL (US)

(21) Appl. No.: 17/166,218

(22) Filed: Feb. 3, 2021

(51) Int. Cl. A61B 5/00; A61B 5/395; A61B 5/107; A61B 5/0531; A61B 5/0205; A61B 5/024; A61B 5/11; A61B 5/16

(52) U.S. Cl. CPC A61B 5/4824; A61B 5/395; A61B 5/1071; A61B 5/0531; A61B 5/0205; A61B 5/0057; A61B 5/1121; A61B 5/4035; A61B 5/7225; A61B 5/743; A61B 5/165; A61B 5/024

(57) ABSTRACT

A method of evaluating rehabilitation potential in patients experiencing painful musculoskeletal or neuropathic conditions or both, by maximizing multidimensional rehabilitation outcomes by better identifying the specific needs of each patient; understanding the many obstacles to rehabilitation success falling into motivational, physical, and emotional categories; physical obstacles to rehabilitation including but not limited to incorrect diagnoses and deconditioning; emotional obstacles including fear avoidance, catastrophizing, generalized anxiety, depression, childhood adverse events, emotional awareness deficits; and motivational obstacles including factitious disorder, personality disorders, malingering and apathy. The resulting comprehensive pain rehabilitation program combines multiple treatment options. Medicinal treatments include anti-inflammatory medications; muscle relaxants; opioids and cannabinoids; injection therapies; neuropathic medications; and pain adjuvant medications. Mental health services include mindfulness, biofeedback, progressive relaxation, cognitive behavioral therapies, logotherapy, and trauma therapies. Physical rehabilitation services include physical or occupational therapy; chiropractic; massage therapy; acupuncture; osteopathy and personalized exercises.

### CROSS-REFERENCE TO RELATED APPLICATIONS

None

### FEDERALLY SPONSORED RESEARCH

None

### BACKGROUND

### Field of the Invention

The invention relates to the general field of rehabilitation potential in painful musculoskeletal and neuropathic conditions. Specifically, the invention relates to maximizing multidimensional protocols for the rehabilitation and pain resolution in patients with chronic painful musculoskeletal and neuropathic conditions.

### Description of the Related Art

The resolution of chronic pain syndromes is a major problem in our society, especially in the current time of the Opioid Crisis. It is estimated that 85% of the Opioid Crisis does not involve what are generally perceived to be “street addicts.” Rather, about 85% of the individuals suffering from opioid addiction in the United States are actually ordinary people who had surgery for such conditions as back surgery, hip surgery, knee surgery, etc. After surgery, they may be initially put on morphine for a few weeks and then put on oxycodone for about two months. Thereafter, they were put on hydrocodone for a period of time.

By the time they are healed, they may become addicted to opioids and when they are taken off opioids, their mind brings back the pain root and they are suffering without the opioids, which they crave due to their addiction. When put back on the opioids, after some period time, the amounts of opioids they were prescribed are not enough and they may be prescribed larger doses of opioids. A significant number end up going from doctor to doctor to obtain more opioids to relieve their chronic pain, which has resulted in a terrible Opioid Crisis in the United States.

It is now well-established that the best approach to manage patient pain is the use of a multidimensional pain rehabilitation program according to the Biopsychosocial Model of Pain (BPSM). The problem of chronic musculoskeletal pain treatment has been extensively analyzed. A 2010 review of the world's literature by the International Association for the Study of Pain found that a multidimensional pain treatment program provides a safer and far more effective approach for the functional rehabilitation of patients than the current biomedical approach of surgeries, anesthetist injections and opioid medications (IASP Fact Sheet No. 7: Evidence-Based Biopsychosocial Treatment of Chronic Musculoskeletal Pain 2010).

Currently, there are both inpatient and outpatient programs. The problem is that many of the best programs are very expensive. The best published results indicate that the pain relief achieved is in the range of 60 to 65%, which is grossly insufficient. As a result, much improvement is needed.

The Biopsychosocial Model of Pain is based on the International Association for the Study of Pain (IASP) definition of “pain” as “an unpleasant sensory and emotional experience associated with actual or potential tissue damage or described in terms of such damage.” This definition explicitly implies that the pain experience has three components including: [a] sensory-physical aspect; [b] an emotional element; and [c] a learned socio-dynamic constituent. Applicant's prior invention “Evaluation of Pain in Humans” U.S. Pat. No. 10,292,640 B2 was the first neurophysiologic assessment of these constituents and their respective interaction.

There is currently no method to refine the Biopsychosocial Model to the area of pain rehabilitation. There are many variations on the rehabilitation techniques including inpatient and outpatient paradigms. A systematic literature review of the world's data on multidimensional pain rehabilitation programs and their published outcomes analyzed 41 separate programs with different but fairly fixed programs, i.e., the same approach for all admitted patients. The published review is available at “Kamper S J, Apeldorn A A T, Chiarotto A T, et al. Multidisciplinary biopsychosocial rehabilitation for chronic low back pain: Cochrane Systematic Review and Meta-analysis. BMJ 2015: 350: h444.”

Current programs report similar outcomes demonstrating that no single method is overall superior. The inference is that “no glove fits all.” The critical problem is therefore to better identify the specific needs of each particular patient. There are many obstacles to rehabilitation falling into motivational, physical, and emotional categories. Some of the physical ones include incorrect diagnoses, refractory pain, deconditioning, and others. Some of the emotional ones include fear avoidance, catastrophizing, generalized anxiety, depression, childhood adverse events, emotional awareness deficits, and others. There are motivational obstacles including factitious disorder, personality disorders, malingering, apathy and others.

Currently, the only mechanism used to distinguish between these mechanisms involve physician/psychologist judgment by interview and questionnaires. These methods are subject to bias and the limitations of self-report. Identifying physiologic biomarkers that better categorize these elements will improve accuracy to the rehabilitation protocols and allow individualized precision medicine intervention. This would be a first of its kind invention because no physiological biomarkers exist for these mostly “psychological concepts.”

Although there are patents in the general field of this patent application, none of the above-listed patents and publications, alone or in any combination, teach or suggest the claimed invention.

There exists a great need in our country to overcome the current problems associated with individuals becoming addicted to opioids.

### SUMMARY OF THE INVENTION

The present invention provides a means of better identifying the needs of the individual patient, based on unequivocal neurophysiologic biomarkers. This allows implementation of more precise rehabilitation programs with greatly improved outcomes.

The cornerstone of functional physical rehabilitation for painful musculoskeletal and neuropathic condition is motion. There are three basic types of movement of a given body region. The three basic types of movement are isotonic, isometric, and isokinetic motions. Isokinetic movements are fairly uncommon in nature, and will not be further described herein. Isotonic movement, as a term, describes movement against a relative constant resistance. Isometric movement describes attempted movement against an immobile obstacle. Negative emotional and sociological factors can interfere with these movements, in different ways. This is the part of the mechanisms by which these movements maintain and exacerbate the pain experience. Additionally, these are the mechanisms by which they interfere with functional pain-related rehabilitation.

Based on neurophysiologic brain principles, the method by which these negative bio-psychosocial mechanisms manifest themselves have different neurophysiologic profiles. The invention for the first time exploits these patterns to classify and identify which mechanisms are operative in a particular individual.

The invention employs four outputs to monitor brain and central nervous system output; these include blood volume pulse, heart rate, skin conductance response for autonomic measurements; and surface electromyography for somatic measurements. These outputs will be monitored before, during, and after the stimulations.

The invention employs two types of inputs; i.e., for isotonic and for isometric movement, at two different levels of intensity. It also compares response under these either, as warranted, inputs in a control region of the body to the same inputs to the responses to the test, i.e., painful body region. Thus, each person acts as his or her own control, mitigating such issues as age, gender, race, concomitant medical conditions, and culture. Each protocol will be performed three times at two different intensity starting with the control site. The first intensity will be the Odynic Identification Threshold (OIT), which is defined as the stimulus intensity when the individual first states that tenderness or discomfort is felt during the motion. The second intensity will be the Odynic Tolerance Level (OTL) which is the maximum movement or exertion the individual is willing to sustain for three seconds. Based on physiologic principles, a maximum discomfort, whether sensory-physical or emotional, should be associated with a robust increase in autonomic phenomena.

### BRIEF DESCRIPTION OF THE DRAWING

FIG. 1 is directed to a first half of a flowchart representing an example of electrode placements for a patient with low back pain, along with data received from electrodes, receipt of data for processing, and both a preamplifier and analog-to-digital conversion of received analog data.

FIG. 2 is directed to the second half of the flowchart representing the receipt and processing of the data of the stimulation (active range of motion during lumbar flexion) and the monitored autonomic and somatic muscles before, during, and after the motion of the patient.

FIG. 3 is directed to a diagram showing a more detailed depiction of surface (EMG) electrodes.

FIG. 4 is directed to a diagram showing the standard placement of the electrodes for autonomic recordings.

FIG. 5 is directed to a representation showing details of the electrode connection to the computer.

FIG. 6 is directed to a graph showing an example of an idealized graph (610) of a sensory-physical graph of a sensory-physical response in a nontender (control) body region.

FIG. 7 is directed to a graph showing a sensory-physical type of pattern in a painful body region (test site).

FIG. 8 is directed to a graph showing a mixed sensory physical and emotional response.

FIG. 9 is directed to a graph showing a mixed sensory physical and emotional response.

FIG. 10 is directed to a graph showing a sociodynamic response.

FIG. 11 is directed to a flow chart showing the application of the invention to the management of an individual patient suffering from chronic musculoskeletal pain.

### DETAILED DESCRIPTION OF THE EXEMPLARY EMBODIMENTS

### Turning to the Figures:

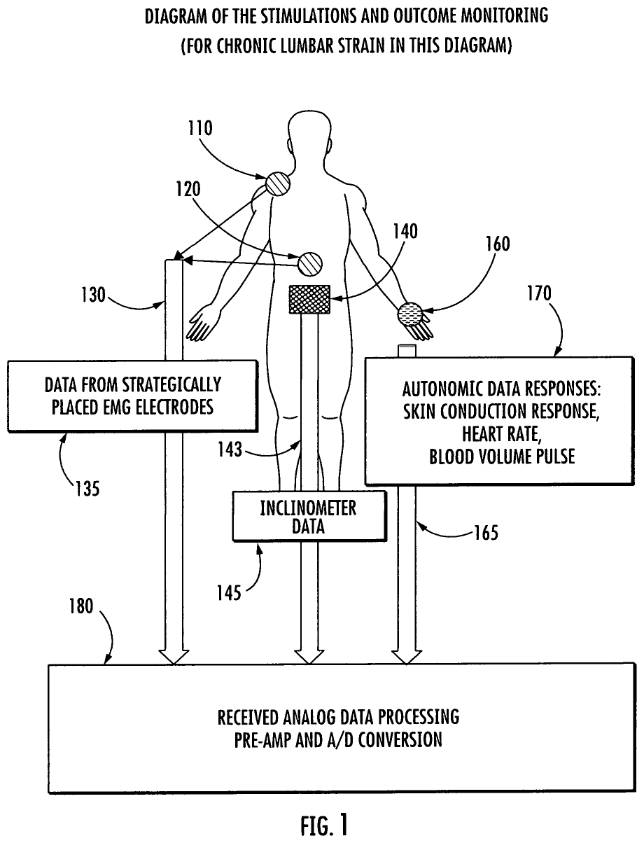

FIG. 1 is directed to a first half of a flowchart representing an example of electrode placements for a patient with lower back pain. The patient will be instructed to actively flex to two levels of pain intensity (OIT and OTL) in separate trials. Labels 110 and 120 depict two sites each of surface electromyographic (sEMG) electrodes to strategically placed to monitor muscle activity during the active range of motion. That analog electrical data will be transmitted to the analog receiving and processing unit 180 by cable transmission (130). Box (135) represents data from the strategically placed electrodes (110 and 120). An inclinometer (140) is placed on the patient's spine, and will measure the angular degrees of change during the active range of motion, in this example lumbar flexion, during the movement trials; that information will be transmitted to the processing unit (180). Box (145) represents analog data being transferred to processing unit (180) via cable (143). The three major autonomic responses, skin conduction response, heart rate, and blood volume pulse, will be monitored by electrodes (160) placed on the fingers of the patient. The data will be transmitted to the processing unit by cables (165). The processing unit (180) will pre-amplify the analog data and convert the amplified analog data to digital data via a pre-amplifier within processing unit (180).

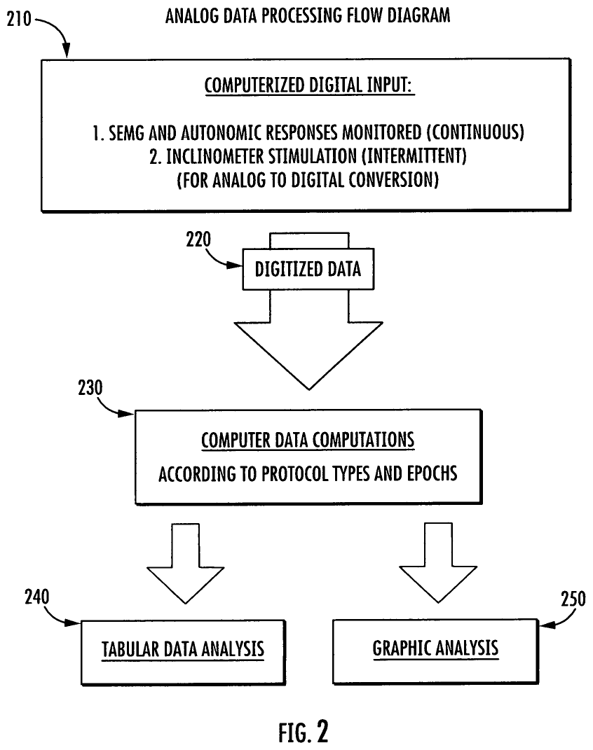

FIG. 2 is directed to the second half of a flowchart representing the processing of the stimulation active range of motion during lumbar flexion and the monitored autonomic and somatic muscles before, during, and after the motion. The analog data is transmitted from the patient via cables (130, 143, and 165) to the pre-amplifier (180) and amplifier (210) where the data is converted to digital data. This digitized data (220) will be transmitted to the computer (230) for analysis. According to specifically designed protocols and time periods, i.e., epochs; the data will be computed comparing the results of the OIT and OTL trials. These computations will be used to determine whether the evoked “pain” is comprised of sensory-physical, emotional, or sociodynamic components. The computations will be presented in tabular (240) and graphic (250) analysis for reader clinical interpretation.

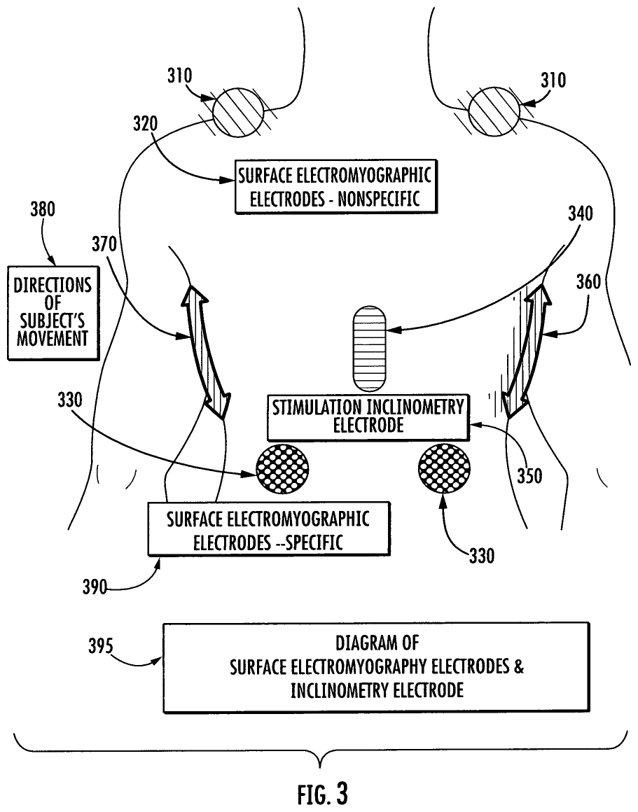

FIG. 3 is directed to a diagram showing a more detailed depiction of surface (EMG) electrodes (310). In this example, the patient's back is depicted. This is a patient with mid-back discomfort. The patient will be asked to rotate or laterally bend the mid-back [thorax] to OIT and OTL levels in the direction that causes maximum pain; see (labels 360 and 370). Two non-specific surface electromyographic (sEMG) electrodes are placed on the trapezius muscles that will not be materially involved in the movement; see labels (310 and 320). Two specifically-placed sEMG electrodes are placed on the paraspinal muscles that will be involved in the movement; see labels (340 and 350). The inclinometer, labels (340 and 350), will be placed over the thoracic spine to measure the angle of deviation. The arrows, labels (360, 370 and 380) show the potential directions of motion. Label 390 refers to specific surface electromyographic electrodes. Moreover, label 395 refers to surface electromyographic electrodes and an inclinometry electrode

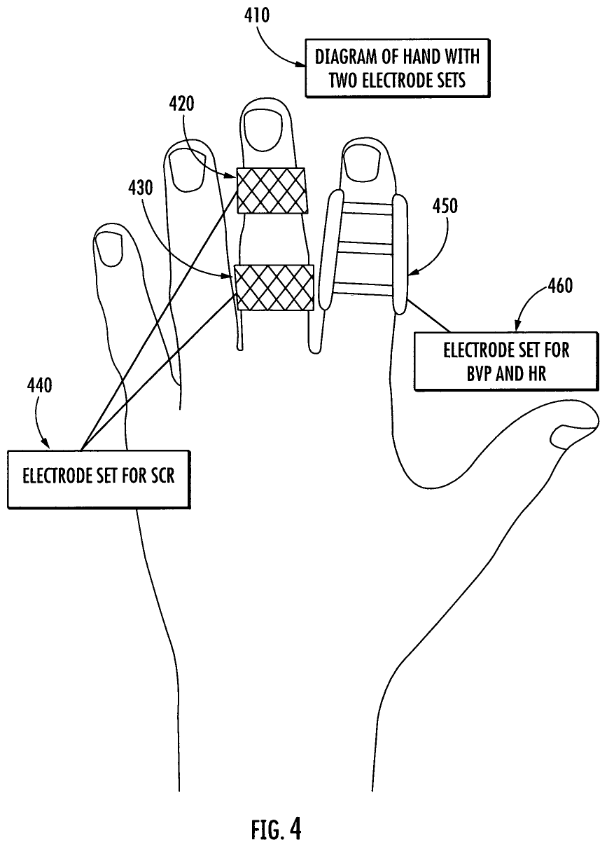

FIG. 4 is directed to a diagram showing the standard placement of the electrodes for autonomic recordings. In this example, the left hand is shown at (410). Either hand can be used, away from the area of stimulation. The electrode set for monitoring skin conduction resistance (SCR) is shown at labels (420, 430 and 440). The unit for measuring both blood volume pulse (BVP) and instantaneous heart rate (BR) is shown at labels (450 and 460). BVP is an analog of finger blood pressure and changes with autonomically mediated vasoconstriction. HR is an analog of the pulse measured at the finger level.

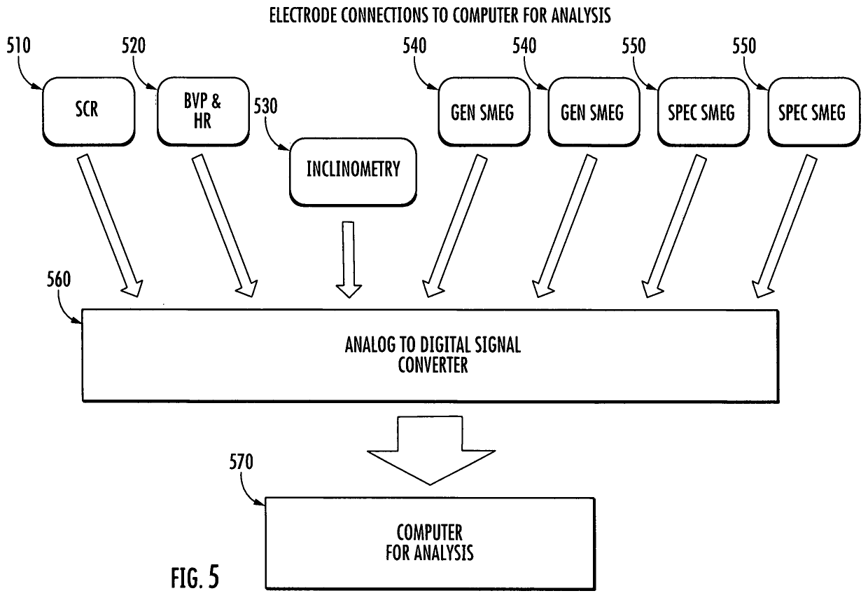

FIG. 5 is directed to a representation showing details of the electrode connection to the computer. Each electrode is processed separately. The SCR (510) and BVP/HR (520) indicate the incipient processing of the patient's autonomic tone data throughout the entire protocol before, during and after the movement. The inclinometer (530) measures the amount of movement only: lasting about 3 seconds for each motion. The general sEMG electrodes (540) indicate the processing of the patient's general muscle tone away from the region of movement. The specific sEMG electrodes (550) indicate the processing of the change in muscle activity by the muscles in the region of movement. All the analog information is transmitted to the analog-to-digital converter/pre-amplifier (560) and then for the computer for data analysis [570].

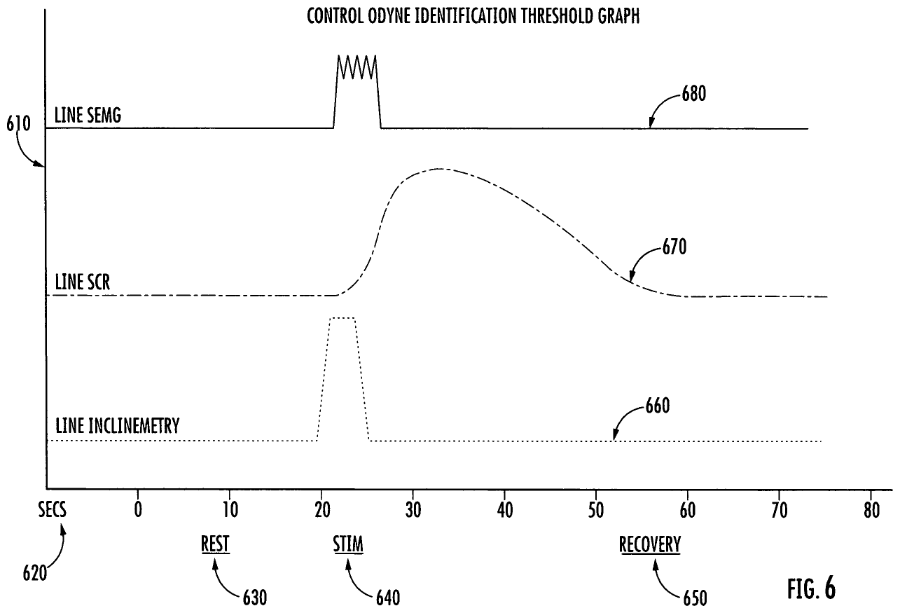

FIG. 6 is directed to a graph showing an example of an idealized graph (610) of a sensory-physical graph of a sensory-physical response in a nontender “control” body region. In this example, the patient is asked to flex the neck to where the motion becomes initially uncomfortable OIT level stimulation. Time in seconds is monitored on the x-axis (620). Various timeframes, epochs are shown; an initial “Rest” epoch (630) before the motion; a “Stimulation” epoch (640) where the patient flexes and then relaxes; and a “Recovery” epoch (650) after which the Stimulation ends; and the patient's nervous system is allowed to recover. For simplicity, only three monitored responses are shown. The measured motion; i.e., inclinometry, is shown depicting the amount of active range of motion required to reach OIT (660). Only the Skin Conductance Response SCR is shown (670); the BVP and FIR are not. The SCR rises and then falls after the mildly “painful” stimulation in response to the stimulation itself. Only one sEMG is shown (680); it is specifically-located, and shows some muscle activation during the stimulation period only.

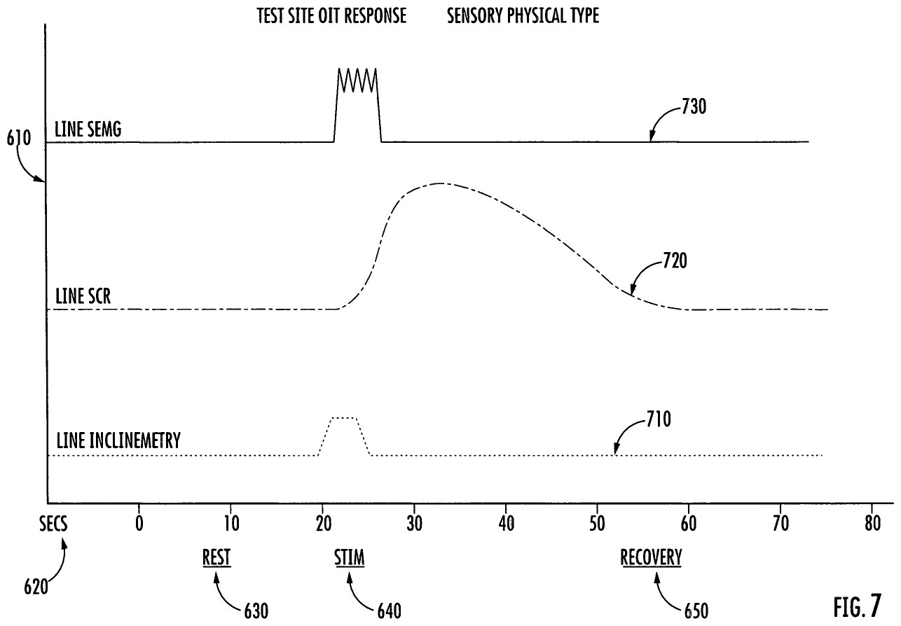

FIG. 7 is directed to a graph showing a sensory-physical type of pattern in a painful body region, at the Test Site. The electrodes, other parameters, and numbering system are similar to FIG. 6 labels (610, 620, 630, 640 and 650). The difference in this idealized graph is that the OIT threshold level (710) is reached at a much-reduced stimulation amount; in this example inclinometry, or active range of motion. The SCR (720) and sEMG (730) tracings are the same as in FIG. 6. The scientific inference is that the Test Site is physically more tender because the brain reacts the same way to the evoking stimulation but at a much lower magnitude of stimulation.

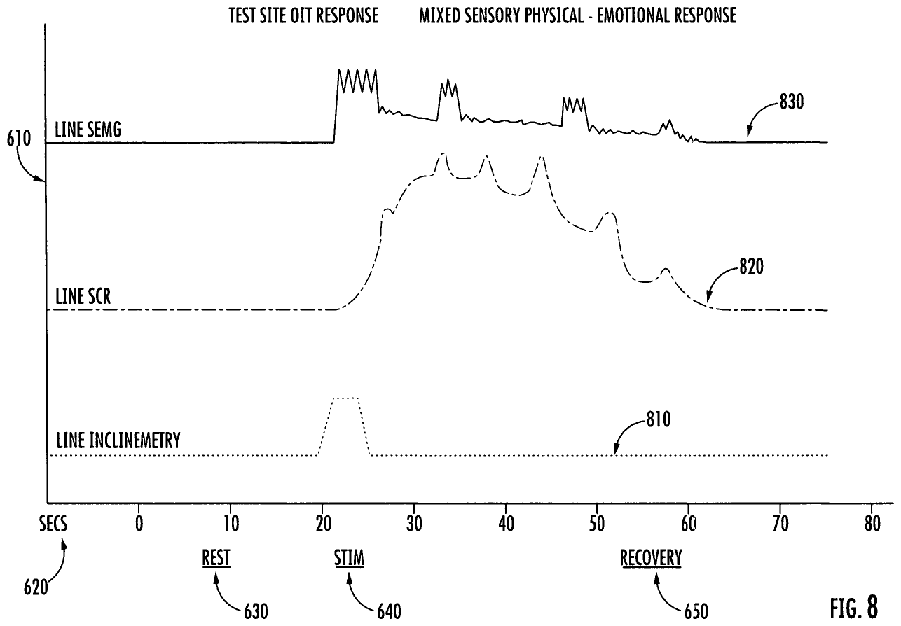

FIG. 8 is directed to a graph showing a mixed sensory physical and emotional response. The electrodes, parameters, and numbering systems are similar to FIG. 6. The difference in this graph is that the Inclinometry level (810) is reduced from the graph of FIG. 6, but higher than the graph of FIG. 7, indicating that OIT threshold for tenderness and nervous system activation is somewhere in-between. The critical difference is that the shape of the SCR (820) and sEMG (830) responses. There are physiologic perturbations of these responses occurring without an external stimulus; i.e., after the stimulation has ended. These independently evoked responses are arising from internal “emotional” phenomena. This reflects a reactive emotional response to the stimulation.

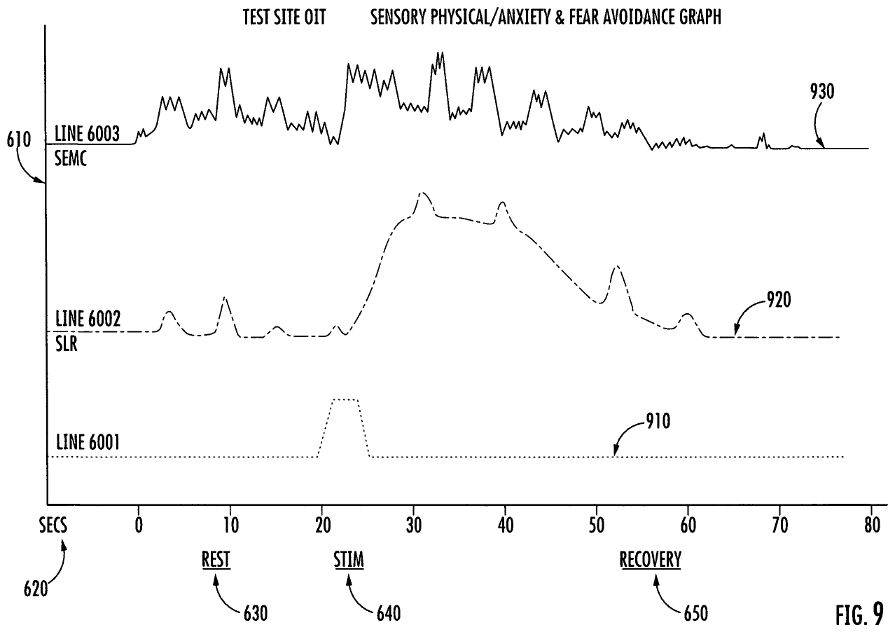

FIG. 9 is directed to a graph showing a mixed sensory-physical/emotional response with fear avoidance. Again, the electrodes, parameters, and numbering system are similar to FIG. 6. The Inclinometry level (910) is the same as in FIG. 8; (between the levels in FIGS. 6 and 7), with the same implications. This time, the SCR graph (920) shows additional SCR changes during the pre-stimulation rest period while maintaining the perturbations during the recovery period. This time, the sEMG graph (930) shows muscle perturbation before the stimulation. This anticipatory physiologic activity is known as fear avoidance plus the previously shown reactive emotional changes.

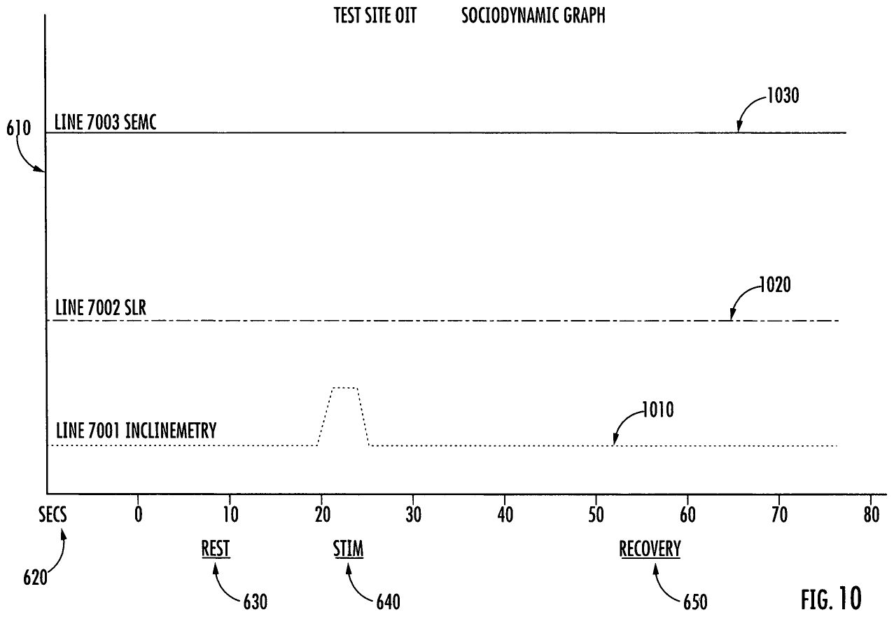

FIG. 10 is directed to a graph showing a socio-dynamic response. Again, the electrodes, parameters, and numbering system are similar to FIG. 6. The Inclinometry level (1010) again is the same is in FIGS. 8 and 9. In this situation, there are no perturbations of the SCR (102) before, during or after the stimulation. In this situation, there are no perturbations of sEMG response (103) before, during, or after stimulation. The implications are that the verbalized “pain” experience is non-physiologic; as it has no neurophysiologic correlates. The verbalized symptom must have a psychological or motivational, i.e., sociodynamic etiology.

with regard to comparing profiles, by comparing data and graphs between control site conditions, the healthcare provider can assess the presence or absence of generalized emotional-social factors of a generalized type that can impede rehabilitation. By comparing data and graphs between control and test site response, one can identify test-site problems that can impede rehabilitation. The types of changes indicated in FIGS. 4 through 7 can occur to different magnitudes and different patterns during the OIT and OTL trials.

These examples show four of the five fundamental response types. These include: [a] the sensory physical type; [b] the reactive emotional response; [c] the anticipatory emotional response [not shown]; [d] the anticipatory and emotional response; and [e] the socio-dynamic response. These can mix in various combinations. The total number of permutations for the OIT stimulation is 15 (fifteen). Similarly; there are fifteen permutations for the OTL stimulation. Combining the two, there are 225 potential permutations for the combined OIT-OTL stimulations. It is this great heterogeneity that confounds current rehabilitation efforts. Providing healthcare professionals more accurate information about a particular patient's needs will vastly improve outcomes because of the greater assessment accuracy and detail.

Chronic musculoskeletal pain is a major health problem in most Western countries. In the United States, the direct costs of medical care is $150 billion annually. The total costs, including disability, lost productivity, and other costs, is estimated at $650 billion per annum. Despite these enormous efforts, musculoskeletal conditions are still the most common cause of work-age permanent disability. Attempts to control the pain have contributed to the prescription Opioid Crisis.

Modern science now proves that the most effective way of treating most cases of chronic musculoskeletal pain is with a multidimensional pain rehabilitation program; according to the Biopsychosocial Model of Pain. The term “Biopsychosocial Model or Pain” refers to the newer, scientifically validated multidisciplinary treatment of chronic pain with a team approach; physical rehabilitation, mental health care, and physician support. In general, the Biopsychosocial Model of Pain; in-patient multidisciplinary approach is far more successful than Biomedical treatments; with a minority of exceptions. BPSM approaches have success rates in the 60% range for return to work, versus 20% for the Biomedical approach. These programs include the combination of [a] medicinal oversight and medications; [b] physical rehabilitative approaches such as physical therapy, occupational therapy, and others; and [c] mental health intervention such as cognitive behavioral therapy, mindfulness, and others. There are many different ways of combining these various options. Because of this, there is no general consensus on how to best manage these musculoskeletal conditions; for example, chronic low back pain, in a particular individual. The physical rehabilitative efforts are often less successful than hoped because psychosocial factors such as depression, anxiety, fear of movement, or motivational problems interfere with adherence and outcomes.

Having a physiological biomarker that would address the physical rehabilitative and mental health needs of a specific individual would allow the patient to get a much more precise rehabilitation program tailored to that person's needs. The Physical Rehabilitation Physiologic Evaluation (PRPE) is uniquely designed to address that critical issue.

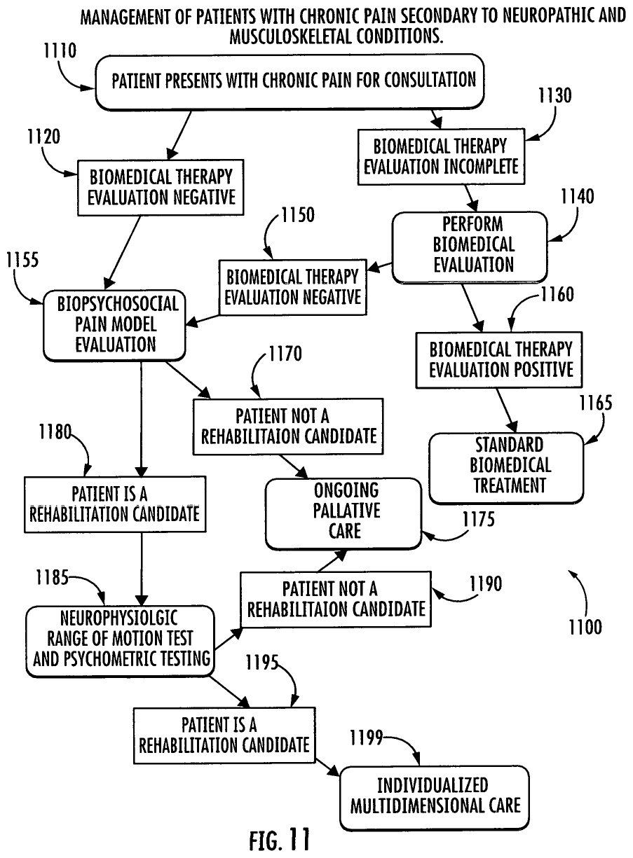

FIG. 11 is directed to a flow chart showing the application of the invention to the management of an individual patient suffering from chronic musculoskeletal pain. It shows a more precise and accurate assessment, resulting in more appropriate patient selection and improved outcomes. The following steps are taken in accordance with FIG. 11.
1. The patient presents for an evaluation of chronic pain (111). There are two possible outcomes. The traditional biomedical evaluation is complete (1120) or incomplete (1130).
2. If the evaluation is incomplete; it will be completed (1140).
3. There are then two outcomes, the evaluation which establishes that the patient has clear prior unrecognized physical problems (1160) that can be addressed by traditional aggressive care (also known as biomedical model of pain); usually anesthetist injections or surgery. In that outcome, the patient is referred to the appropriate standard biomedical treatment (1165). The term “Biomedical Treatment” refers to traditional aggressive care with:
   musculoskeletal or neuropathic surgery.
   anesthesiologist injections, such as epidural injections, insertable stimulators.
   chronic opioid therapy.
4. Using low back pain as an exemplar, this might include epidural injections, facet injections, and others. Surgeries might include lumbar laminectomy or fusion; with or without instrumentation; and spinal cord stimulation.
5. The second outcome of the completed consultation of chronic pain is that the patient is not a good candidate for aggressive biomedical care (1120) and (1150).
6. The patient is then referred for a Biopsychosocial Pain Model Evaluation (1155). There are two outcomes.
7. One outcome might be that the patient is not a candidate for a pain rehabilitation program (1170). In that case, the patient is referred for safe ongoing palliative care (1175) [for example, opioid therapy, intrathecal morphine pumps, or other medicinal pain-oriented care and the evaluation process is completed.
8. If the patient is a potential for comprehensive pain rehabilitation (1180), that individual receives a full neurophysiologic and psychological assessment to include the current invention, i.e., “Rehabilitation Potential Physiologic Assessment” (1185).”
9. The results of the comprehensive evaluation will be evaluated There are two potential outcomes of the completed evaluation.
10. In the first potential outcome, the patient is not a rehabilitation candidate (1195) and is referred to a multidimensional chronic pain rehabilitation program (1199) individually tailored to meet the patient's physiologically and psychologically identified needs. These may combine in varying quantities. Below is a list of the major categories for rehabilitation with examples of the subtypes of treatments in each category. The list is not meant to be exhaustive but does provide some notion of the many choices available.
   I. Medicinal pain-oriented care:
      a) Anti-inflammatories/muscle relaxers to reduce inflammation and reduce tenderness.
      b) Muscle relaxers to reduce muscle spasm and tenderness.
      c) Trigger point injection therapies to reduce localized muscle tenderness and spasm.
      d) Joint injections to reduce joint inflammation.
      e) Pain adjuvants.—medications that work on brain pathways that alter central nervous system processing of pain signals.
      f) Other medications such as opioids and cannabinoids.
   2. Physical Rehabilitation
      a) Modalities to reduce muscle spasm and joint inflammation.
      b) Therapeutic Exercises to improve range of motion and muscle tone in a given body region.
      c). Kinetic Exercises to improve coordinated movements and body function.
      d) Home Exercise Program.
      e). Massage, Chiropractic, Acupuncture, Osteopathy or other alternative techniques.
      f) Other.
   3. Mental Health Interventions
      a) Cognitive Behavioral Therapy to change a patient's perceptions of relevant ideas or behavior that interfere with rehabilitation.
      b). Mindfulness—for general relaxation by staying in the calm moment and reducing distractions especially of a negative or emotional type.
      c) Progressive Relaxation—techniques for localized or generalized muscle tightness.
      d). Logotherapy for deeper psychological problems as a search for life meaning.
      e) Others

Currently, the choice of treatment is made by the healthcare professional's judgment and experience in combination with the patient's presentation. This is very prone to imprecision due to patient perception and expectations and provider bias-training. This imprecision explains why multidisciplinary programs often do not succeed (for return-to-work; the failure rate is above 35%).

The Neurophysiologic Range of Motion Test (NPRMT) and psychometric testing provide unprecedented clarity about the patient's rehabilitation needs and attitudes toward the rehabilitation process, which invariably requires improved movement and effort to restore musculoskeletal function. The following examples will provide illustrations of how therapy can be guided by results in patients with chronic low back pain. We will assume three simple patient types presenting with disabling pain.

Predominant Sensory Physical Profile Myofascial Tenderness. The NPRMT reveals substantively tender myofascial discomfort that increases with motion; there is no significant emotional component. The medications will be geared for control of inflammation and muscle spasms; trigger point injections can be used to reduce focal muscle spasms. This will be supplemented by physical therapy aimed at modalities and therapeutic exercises with a home exercise program for regional and generalized conditioning. The mental health interventions would be geared at progressive muscle relaxation and education.

Predominant Emotional Profile. The profile identifies little tissue tenderness but a patient with anxiety/depression increased dramatically by movement anticipation and reaction. The medications will be aimed at mild “pain control” with intermittent anti-inflammatory medications plus anxiolytic and depressive medications; i.e., pain adjuvants; trigger point injections will not be used. The physical therapy will be aimed at therapeutic and kinetic exercises of a progressive type supplemented by guided-increasing home exercises where modalities would not be emphasized. The mental health services would be comprised of mostly of mindfulness, progressive muscle relaxation, including motion-related cognitive behavioral therapy would likely be used.

Predominantly Sociodynamic Profile. The patient's profile reveals minimal tenderness and a suppressed emotional profile with decreased range of motion but no anticipation or reaction. Psychological interview reveals that the patient was a victim of abuse; e.g., child abuse or rape. The medications would be aimed at over-the-counter medications for pain; e.g., acetaminophen and pain adjuvants with antidepressants and anxiolytics as warranted. The physical rehabilitation would emphasize kinetic activities and alternative approaches, such as Yoga or Tai Chi. The mental health services would involve logotherapy and trauma psychology which is possible for any post-traumatic stress.

The reductionist biomedical approach to chronic pain which emphasizes MRI findings and nerve studies has been proven invalid. It does not adequately evaluate the complex nature of the human pain experience. Chronic pain syndromes (especially musculoskeletal or neuropathic ones) are extremely heterogeneous and nuanced. The current NPRMT invention provides the means to accurately differentiate many pain rehabilitative subtypes with a neurophysiological and scientifically valid analysis. That precision improves the rehabilitation algorithm and design that can now be designed with great clarity aimed at the particular patient's individualized needs and rehabilitation experience.

Although one or more exemplary embodiments have been shown or described, other exemplary embodiments would be readily apparent to those of ordinary skill in the art. As a result, the invention is not intended to be limited by the one or more exemplary embodiments, but rather by the metes and bounds of the appended claims.

1. A method of evaluating rehabilitation potential in patients experiencing painful musculoskeletal or neuropathic conditions or both, by maximizing multidimensional protocols for rehabilitation and pain resolution by better physiologically identifying the specific needs of each particular patient; understanding the many obstacles to rehabilitation which fall into motivational, physical, and emotional categories; physical obstacles to rehabilitation including but not limited to incorrect diagnoses, refractory pain, and deconditioning; emotional obstacles to rehabilitation including fear avoidance, catastrophizing, generalized anxiety, depression, childhood adverse events, emotional awareness deficits; and motivational obstacles to rehabilitation including factitious disorder, personality disorders, malingering and apathy; the method comprising the steps of:
a. evaluating a referred patient complaining of chronic pain and determining whether prior traditional biomedical assessments are complete or incomplete;
b. finding the prior traditional biomedical evaluation incomplete due to missing tests that were not performed; completing the missing tests by performing the missing tests which are imaging tests, laboratory tests, and neurodiagnostic test;
c. reviewing the results of the missing tests and identifying a previously unidentified physical problem, providing the patient with traditional biomedical care including anesthetist injections or surgery, and obtaining ail of the results of the completed traditional biomedical care testing;
d. contemplating treatment for low back pain, options include provide epidural or medial branch anesthetist injections; placement of spinal cord stimulator; lumbar surgeries including one or more of foraminotomy, laminectomy, fusion without instrumentation, fusion with instrumentation and others;
e. evaluating the referred chronic pain patient test results and finding prior traditional biomedical work-up completed; determining the patient is a potential candidate for multidimensional pain rehabilitation program that integrates medicinal, psychological and physical rehabilitation; providing the patient with an evaluation according to the Biopsychosocial Model of Pain and obtaining the results of the testing after the testing is completed
f. reviewing the results of the initial Biopsychosocial equation and determining that the patient is not a good candidate for multidimensional pain rehabilitation; the patient will be referred for ongoing chronic pain management including one or more of opioid medication, intrathecal morphine, and intermittent injections therapy;
g. reviewing the results of the initial Biopsychosocial Pain Model and determining the patient is a potentially good candidate for multidimensional rehabilitation; the patient is referred for a detailed, neurophysiologically based, scientifically valid Rehabilitation Neurophysiologic Assessment supplemented by psychological testing which identifies the patients individual needs within a multidimensional pain rehabilitation optimizing the amounts and types of medications, physical rehabilitation options, and mental health therapy, which are provided to the patient; and
h. reviewing the results of the Rehabilitation Neurophysiologic Assessment and supplemental psychological testing, and determining the patient is a good rehabilitation candidate; using the test data results, the patient is put into a specifically designed program that meets the patient's specifically identified neurophysiological and psychological requirements with improved outcomes and reduced overall costs.

2. The method of evaluating rehabilitation requirements for patients experiencing painful musculoskeletal or neuropathic conditions or both, of claim 1, further comprising the steps of:
a. reviewing the Rehabilitation Neurophysiologic Assessment and supplemental psychological testing results which determines the precise type of medication needs of the individual with treatment options for the patient being anti-inflammatory medications reducing inflammation related tenderness; muscle relaxants reducing muscle tensions; trigger point injection reducing focal muscle spasms; Joint injections treating joint tenderness; gain adjuvants that treat brain pain pathway signals; and opioids or cannaboids blocking different pain signals.

3. The method of evaluating rehabilitation potential in patients experiencing painful musculoskeletal or neuropathic conditions or both, of claim 1, further comprising the steps of:
a. reviewing the Rehabilitation Neurophysiologic Assessment and supplemental psychological testing results and determining the precise type of physical rehabilitation needs of the patient including modalities of hot/cold, ultrasound, electric muscle stimulation; neuromuscular re-education; therapeutic exercises improving range of motion and muscle tone of a given body region; kinetic exercises which improve joint movement or extremity motion; muscle relaxants reducing, muscle tensions; and alternatives including chiropractic, massage, manual manipulation, and osteopathy; provided to the patient.

4. The method of evaluating rehabilitation potential in patients experiencing painful musculoskeletal or neuropathic conditions or both, of claim 1, further comprising the step of:
a. reviewing the Rehabilitation Neurophysiologic Assessment and psychological testing results determining the precise types of mental health services required to meet individual patent's needs, with options including Cognitive Behavioral Therapy which changes the patients perceptions of relevant ideas or behavior interfering with rehabilitation; mindfulness improving general relaxation by staying in a calm moment and reducing distractions, especially distractions of a negative or emotional kind; progressive relaxation techniques localizing general muscle tightness; logotherapy treating deeper psychological problems in a search for personal life meaning; trauma therapy alleviating post-traumatic stress disorder; treating childhood adverse experiences; alternative therapies including biofeedback, neurofeedback; and deep meditation.

5. The scientific method of determining the accurate individualized rehabilitation needs for appropriate patients undergoing a multidimensional rehabilitation program for patients experiencing chronic musculoskeletal or neuropathic pains of claim 1, the method comprising the step of:
determining the individual's unique rehabilitation profile from the more than 200 distinct kinds of neurophysiologic and psychological responses evoked during a multidimensional program and its therapies.

6. The method of evaluating rehabilitation potential in patients experiencing painful musculoskeletal or neuropathic conditions or both, of claim 1, further comprising the steps of:
a. reviewing the Rehabilitation Neurophysiologic Assessment and supplemental psychological testing results performed on patients with neuropathic conditions and determining whether the patient is a candidate for a multidimensional pain rehabilitation program;
b. determining that a patient is not a multidimensional rehabilitation program candidate, and providing the patient with long term pain management;
c. determining that a patient is a candidate for a multidimensional rehabilitation program; providing the patient with a scientifically and physiologically-determined medicinal/psychological/rehabilitative testing specifically tailored to meet that individual's specific and personal needs from over 200 hundred possible outcomes.

7. The method of evaluating rehabilitation potential in patients experiencing painful musculoskeletal or neuropathic conditions or both, of claim 1, further comprising the steps of:
a, obtaining data for a patient with chronic lumbar strain from several strategically placed EMG electrodes continuously recording physiological muscle contraction before, during, and after rehabilitation efforts under two or more different conditions relevant to rehabilitative programs based upon pain initial detection and pain tolerance in one or more planes or directions of movement;
b. obtaining data for a patient with chronic lumbar strain from electrodes recording Blood Volume Pulse monitors continuously recording blood vessel tonality before, during, and after rehabilitation efforts under two or more different conditions relevant to rehabilitative programs directed to pain initial detection and pain tolerance in one or more planes or directions of movement;
c. obtaining data for a patient suffering from chronic lumbar strain, the data is obtained from electrodes recording Skin Conductance Response from monitors continuously recording body sudomotor tonality before, during, and after rehabilitation effort; under two or more different conditions relevant to rehabilitative programs with respect to pain initial detection and pain tolerance in one or more planes or directional movement;
d. obtaining data for a patient with chronic lumbar strain; the data being obtained from electrodes recording instantaneous heart rate monitors continuously recording cardiovascular autononic tonality before, during, and after rehabilitation efforts obtained under at least two or more different conditions relevant to rehabilitative programs based upon pain initial detection and pain tolerance in one or more planes or directions of movement;
e. obtaining all the above data from a patient with chronic pain using different modes of stimulation relevant to pain rehabilitation; including range of movement measures using inclinometry; different rates of movement with time measured inclinometry; tissue tenderness to physical stimulation using algometry; and neuropathic tenderness using direct current stimulation.

8. The method of evaluating rehabilitation potential in patients experiencing painful musculoskeletal or neuropathic conditions or both, of claim 7, further comprising the step of:
a. providing electrode connections to the patient which obtains data relating to one or more of SCR, BVP & HR, inclinometry, GEN sEMG, and SPEC sEMG.

9. The method of evaluating rehabilitation potential in patients experiencing painful musculoskeletal or neuropathic conditions or both, of claim 8, further comprising the steps of:
a. receiving the analog data and converting the analog data to digital data using both a pre-amplifier and a analog-to-digital converter.

10. The method of evaluating rehabilitation potential in patients experiencing painful musculoskeletal or neuropathic conditions or both, of claim 9, further comprising the step of:
a. using the obtained digital data as part of obtaining computer computation according to protocol types and epochs, and outputting both of tabular data analysis and graphic analysis.

11. The method of evaluating rehabilitation potential in patients experiencing painful musculoskeletal or neuropathic conditions or both, of claim 10, further comprising the step of:
a. using baseline stimulation and response data which graphically displays results of odynic identification threshold stimulation, SEMG responses, SCR responses, BVP responses, HR response from rest periods, and a stimulation period and a recovery period.

12. The method of evaluating rehabilitation potential in patients experiencing painful or neuropathic conditions or both, of claim 11, further comprising the steps of:
a. using test level stimulation and response data which graphically displays results of pain tolerance stimulation; SEMG responses, the SCR response, the BVP response, and HR response from rest periods, the stimulation period and the recovery period.

13. The method of evaluating rehabilitation potential in patients experiencing painful musculoskeletal or neuropathic conditions or both, of claim 10, further comprising the steps of:
a. comparing the graphic data and tabulated data between baseline control and test level stimulations from odynic identification threshold stimulations, in determining the core type of individual baseline pain experience, of which there are many types; primary profiles include:
a. predominant sensor physical profile;
b. predominant emotional profile for which there are three types;
i. predominant anticipatory emotional profile;
ii. predominant reactive emotional profile;
iii. predominant mixed emotional profile;
c. predominant sociodynamic profile: with different combinations of these primary types; there are 15 variations or subtypes for the odynic identification threshold stimulation protocol.

14. The method of evaluating rehabilitation potential in patients experiencing painful musculoskeletal or neuropathic conditions or both, of claim 10, further comprising the step of:
using control site stimulation and response data to graphically display results of patient odynic tolerance level stimulation, sEMG responses, the SCR response, the BVP response, and HR response from rest periods, the stimulation period and the recovery period.

15. The method of evaluating rehabilitation potential in patients experiencing painful musculoskeletal or neuropathic conditions or both, of claim 10, further comprising the steps of:
[a] using Test Site stimulation and response data to graphically display results of odynic tolerance level stimulation, SEMG responses, the SCR response, the BVP response, and HR response from rest periods, the stimulation period and the patient recovery period.

16. The method of evaluating rehabilitation potential in patients experiencing painful musculoskeletal or neuropathic conditions or both, of claim 13, further comprising the steps of:
[a] comparing the graphic data, and tabulated data, between paired and test sites from the odynic tolerance level stimulations, determining the secondary-type of individual baseline pain experience of which there are many types; major secondary profiles include:
a. predominant sensory-physical profile;
b. predominant emotional profile for which there are three types;
i. predominant anticipatory emotional profile;
ii. predominant reactive emotional profile;
iii. predominant mixed emotional profile; and
c. predominant sociodynamic profile; with different combinations of these primary types, there are 15 variations or subtypes for the odynic tolerance level stimulation protocol.

17. The method of evaluating rehabilitation potential in patients experiencing painful musculoskeletal or neuropathic conditions or both, of claim 13, further comprising the step of:
[a] comparing the results from the odynic identification threshold paired protocol with the results from the odynic tolerance level paired protocol; determining how the patient adjusts to different intensities of the pain experience, of which there are over 200 different possible combinations.

18. The method of evaluating rehabilitation potential in patients experiencing painful musculoskeletal or neuropathic conditions or both, of claim 10, further comprising the steps of:
[a] using different stimulation modalities to evoke odynic identification threshold and odynic tolerance threshold, depending on rehabilitation needs, of which there are three basic types:
a. range of motion,
i. active range of motion, the most commonly used;
ii. passive range of motion;
b. algometry, and
c. electric current stimulation.

19. The method of evaluating rehabilitation potential in patients experiencing painful musculoskeletal or neuropathic conditions or both, of claim 17, further comprising the step of:
[a.] comparing the outcome of the odynic identification threshold to odynic tolerance level comparison with psychometric data; finding the best personalized rehabilitation program for the patient.

20. A scientific-measurable Neurophysiological Method of evaluating rehabilitation potential in patients experiencing painful musculoskeletal or neuropathic conditions or both, thereby maximizing multidimensional protocols for rehabilitation and pain resolution: by better identifying the specific needs of each particular patient; understanding the many obstacles to rehabilitation which fall into motivational, physical, and emotional categories; physical obstacles to rehabilitation including but not limited to incorrect diagnoses, refractory pain, and deconditioning; emotional obstacles to rehabilitation including fear avoidance, catastrophizing, generalized anxiety, depression, childhood adverse events, emotional awareness deficits; and motivational obstacles to rehabilitation including factitious disorder, personality disorders, malingering and apathy; the method comprising the steps of:
a. determining whether a referred patient has completed or not completed a traditional biomedical evaluation of chronic musculoskeletal or neuropathic pain symptoms;
b. completing the traditional biomedical evaluation after determining that the evaluation was not complete;
c. finding a previously unidentified biomedical problem; patient referred to the appropriate specialist for definitive traditional care;
d. finding that the patient is not a good candidate for traditional biomedical care, the patient is referred for an initial Biopsychosocial Pain Model rehabilitation evaluation;
e. performing the initial Biopsychosocial Pain Model rehabilitation evaluation and finding that the patient is not good rehabilitation candidate, patient referred to chronic palliative pain management;
f. the Biopsychosocial Pain Model Evaluation leads to one or two outcomes; performing the initial Biopsychosocial Pain Model rehabilitation evaluation and finding that the patient is a good rehabilitation candidate, patient obtains a more detailed evaluation which accurately determines that the patient's multidimensional rehabilitation requirements;
g. performing a scientific, neurophysiologically-based Rehabilitation Neurophysiologic Assessment supplemented by a psychological evaluation, the results are reviewed by the multidimensional rehabilitation team leader;
h. reviewing the results of the Rehabilitation Neurophysiologic Assessment supplemented by the psychological evaluation, the patient determined to not be a rehabilitation candidate; and patient referred for chronic palliative pain management;
i. reviewing the results of the Rehabilitation Neurophysiologic Assessment supplemented by a psychological evaluation, the patient determined to be a good rehabilitation candidate and referred for multidimensional rehabilitation with accurate specific details to meet that patient's needs.

21. A method of evaluation rehabilitation potential in patients experiencing musculoskeletal painful musculoskeletal or neuropathic conditions or both, to maximize outcomes from multidimensional pain rehabilitation and resolution protocols by better identifying each particular person's needs in three necessary areas relevant to rehabilitation success while identifying potential impediments, biophysical problems including incorrect diagnoses, refractory physical pain, deconditioning; emotional obstacles to rehabilitation including fear avoidance, generalized anxiety, depression, childhood events, and emotional awareness difficulties; and negative motivational obstacles to rehabilitation including factitious disorder, personality disorders, psychotic psychiatric diagnoses, malingering, apathy; and sociodynamic factors that cannot contribute to treatment failure if unidentified; the method comprising the steps of:
a. determining whether the referred patient's prior traditional biomedical evaluation was complete or incomplete;
b; finding the prior traditional biomedical work-up incomplete, completing the work-up; a patient with previously unidentified biomedical problems referred for definitive for definitive traditional biomedical care;
c. finding the prior traditional biomedical work-up incomplete, the work-up is completed; a patient with no unidentified issues is referred to an initial Biopsychosocial Pain Model evaluation; biophysical, emotional, and motivational/sociodynamic factors are assessed;
d. performing the initial Biopsychosocial Pain Model evaluation and determining that the patient is not a good candidate for a multidimensional Biopsychosocial pain rehabilitation program, patient referred for chronic palliative pain management;
e. performing the initial Biopsychosocial Pain Model evaluation and determining that the patient is a good candidate for a multidimensional Biopsychosocial Pain rehabilitation program, patient referred for a detailed neuroscientifically valid Rehabilitation Neurophysiologic Assessment supplemented by psychological assessment;
f. performing the neuroscientifically valid Rehabilitation Neurophysiologic Assessment and supplemental psychological testing, patient determined to not be a rehabilitation candidate is referred to chronic palliative pain management;
g. performing the neuroscientifically valid Rehabilitation Neurophysiologic Assessment and supplemental psychological testing, the patient e determined to be a good rehabilitation candidate and is referred to a multidimensional pain rehabilitation with the test used to design and implement an individualized multidimensional program according to the tenets of precision medicine to maximize a successful outcome; treatment needs of specifically identified biophysical, emotional, and sociodynamic problems are managed.

22. A method for the evaluating the patient potential rehabilitation of a patient suffering from neuropathic or musculoskeletal or combined conditions; scientifically identifying a patient's specific pain rehabilitation needs according to the Biopsychosocial Model of Pain; the testing provides unprecedented data concerning the biophysical, emotional, and sociodynamic factors combining to create chronic pain; using that information to design and implement an individualized multidimensional rehabilitation to maximize a successful outcome; the method compromising the steps of:
a. determining whether the referred patient's prior traditional biomedical work-up was complete or incomplete;
b. finding the prior traditional work-up incomplete, the work-up is completed; and finding any previously unidentified biomedical conditions, referral for definitive traditional biomedical treatment is made;
c. finding the prior traditional work-up complete, the patient is initially assessed for multidimensional rehabilitation according to the Biopsychosocial Model of Pain, and determined to not be a good rehabilitation candidate; the patient is referred for chronic palliative pain management;
d. finding the prior traditional work-up complete, the patient is initially assessed for multidimensional rehabilitation according to the Biopsychosocial Model of Pain, and determined to be a good rehabilitation candidate; the patient e, undergoes the scientifically valid Rehabilitation Physiologic Assessment and psychometric testing;
f. performing the Rehabilitation Neurophysiologic Assessment supplemented by psychological evaluation and reviewing the data, the person will be determined not to be a good candidate for multidimensional rehabilitation program and is referred for chronic palliative pain management;
g. performing the Rehabilitation Neurophysiologic Assessment supplemented by psychological evaluation and reviewing the data; the patient is determined to be a good candidate for multidimensional pain rehabilitation, the data from above assessments are used to create and implement a patient-specific, individualized rehabilitation program.

23. A method for the evaluating the patient potential rehabilitation of a patient suffering from neuropathic or musculoskeletal or combined conditions the method compromising the steps of:
a. determining whether the referred patients prior traditional biomedical work-up was complete or incomplete
b. finding the prior traditional work-up incomplete, the work-up is completed; and finding any previously unidentified biomedical conditions, referral for definitive traditional biomedical treatment is made;
c. finding the prior traditional work-up complete, the patient is initially assessed for multidimensional rehabilitation according to the Biopsychosocial Model of Pain, and determined to not be a good rehabilitation candidate; the patient is referred for chronic palliative pain management;
d. finding the prior traditional work-up complete, the patient is initially assessed for multidimensional rehabilitation according to the Biopsychosocial Model of Pain,
e. determining the patient to be a good rehabilitation candidate; the patient undergoes the scientifically valid Rehabilitation Physiologic Assessment and psychometric testing;
f. performing the Rehabilitation Neurophysiologic Assessment supplemented by psychological evaluation and reviewing the date, the person is determined not to be a good candidate for multidimensional rehabilitation program and is referred for chronic palliative pain management;
g. performing the Rehabilitation Physiologic Assessment supplemented by psychological evaluation and reviewing the data; the patient is determined to be a good candidate for multidimensional pain rehabilitation, the data from above assessments is used to create and implement a patient-specific, individualized rehabilitation program;
